(19) United States
(12) Patent Application Publication
(10) Pub. No.: US 2026/0360541 A1
(43) Pub. Date: Nov. 12, 2026

(54) METHOD FOR ASSESSING THE SEVERITY OF A SLEEP DISORDER

(71) Applicant: Université de Mons, Mons (BE)

(72) Inventor: Luca LA FISCA, Morlanwelz (BE)

(73) Assignee: Université de Mons, Mons (BE)

(21) Appl. No.: 19/475,349

(22) PCT Filed: Apr. 17, 2024
(86) PCT No.: PCT/EP2024/060379

§ 371 Date: Oct. 14, 2025

(30) Foreign Application Priority Data
Apr. 17, 2023 (EP) 23168189.1

(51) Int. Cl. G16H 50/20; G06N 3/0455; G06N 3/094

(52) U.S. Cl. CPC G16H 50/20; G06N 3/0455; G06N 3/094

(57) ABSTRACT

Methods and systems for explainable machine learning are described. In an example, a processor can receive a biosignal data biosignal data associated with a plurality of trials representing different levels of severity of the sleep disorder, wherein the biosignal data includes a plurality of severity features variably impacting the severity of the sleep disorder. The processor can encode the biosignal data from the trials into a plurality of latent vectors characterizing the severity of the sleep disorder, utilizing the severity features, and arranging the latent vectors in a latent space to increase discriminability between latent vectors representing different levels of severity. The processor can operate the machine learning model on biosignal data obtained from a subject to generate a severity score indicative of the severity of the sleep disorder of the subject, determined from a position of the corresponding latent vector in the latent space relative to the plurality of latent vector, and further generate a report comprising at least the severity score that can be used for assessing the severity of the sleep disorder of the subject.

### FIELD OF THE INVENTION

The present disclosure relates to artificial intelligence, and in particular, to an explainable machine learning system that is trained based on biosignal data acquired by one or more biosignal sensors of a sleeping subject, that includes a plurality of features associated with a sleep disorder.

### BACKGROUND

Objective measures of sleep can be obtained by performing a trial such as polysomnography (PSG). PSG is the standard for diagnosing sleep disorders, such as Obstructive Sleep Apnea and hypopneas (OSAs) and other sleep disorders. During PSG, numerous monitoring devices are connected to a sleeping subject, allowing overnight measurement of various biological/physiological parameters. These parameters may include, for example, airflow, heartrate, various electrical and nonelectrical signals, and so on. The biosignal data obtained from these measurements can be used by healthcare professionals to diagnose the sleep disorder and determine the causes of sleep disturbance.

In all these scenarios, the monitoring modalities generate an ever-increasing amount of data which needs to be interpreted. Therefore, predictive models that can classify, detect and ultimately “score” the trial are required. Traditionally, this type of modelling has mostly relied on human interpretation, where large datasets are manually reviewed and annotated by a clinician or trained technician. However, human review is expensive, time-consuming and inherently suffers from bias of the person interpreting the available data. Therefore, it has been difficult to establish a consensus towards a metric for scoring the severity of sleep disorders.

For example, the assessment of how an Obstructive Sleep Apnoea Syndrome affects a subject's health, that is, its “severity”, is typically determined based on the number disturbances occurring overnight through the Apnea-Hypopnea Index (AHI). However, the use such metrics has been largely criticized over the last decade as it fails to estimate the impact of a sleep disorder on related medical conditions. This issue has triggered many works aiming to find better metrics to characterize the severity, such as hypoxic burden, arousal intensity, duration of apnoeic events, odds ratio product, heart rate variability, cardiopulmonary coupling, and so on. Despite efforts made in the direction of identifying the most efficient metric for OSAs severity scoring, no consensus has been found across the medical and sleep research community.

Machine learning (ML) applications such as deep learning models have seen a growing interest in the research domain. It is understood that the use of ML algorithms presents an interesting opportunity to improve the diagnosis of sleep disorders and better the understanding of biosignal data. Nonetheless, due to the black-box nature of ML models, the interpretation of latent spaces may be unintuitive. The latent space may be high-dimensional, complex, and nonlinear, which may add to the difficulty of interpretation. Although various predictive ML models have been explored, it has thus far mostly relied on fully supervised approaches, where large datasets of annotated examples are provided to train models with high performance based on a human-selected metrics.

For example, Feng Kaicheng et al in IEEE Transactions on Instrumentation and Measurement, vol. 70, pp. 1-12 (2020) describes a sleep apnea detection method based on unsupervised feature learning and single-lead electrocardiogram.

Li Kunyang et al. in Neurocomputing, vol. 294, pp. 94-101 (2018) describes a method to detect sleep apnea based on deep neural network and hidden Markov model using single-lead ECG signal.

Kuo Chih-en et al. in Computers in Biology and Medicine, vol. 148, 105828 (2022) described a method related to precision sleep medicine using self-attention GAN as a data augmentation technique for developing personalized automatic sleep scoring classification.

McCloskey Stephen et al. advances in knowledge discovery and data mining, PAKDD 2022, pp. 314-325 describes a method for insomnia disorder detection using EEG sleep trajectories.

However, as mentioned earlier, scoring the sleep disorder severity of a trial solely on the metrics selected by a person annotating the training data introduces bias into the final score. Therefore, the quality of supervised learning paradigms is often limited by the amount of labelled data that is available and the reliability of metrics that is used to train the model. This phenomenon is particularly problematic when working with clinically relevant data, where the model will simply propagate the bias introduced by the learned metrics. Consequently, deep learning architectures designed to severity score a trial solely on a pre-identified metric yield relatively shallow models and performances that are at best similar to those of traditional feature-based approaches.

Therefore, a new framework that does not rely primarily on subjective bias that metrics proposed by the medical and sleep community suffer from is necessary to help identify and severity score a trial of importance for a sleep disorder severity assessment task.

### SUMMARY OF THE INVENTION

As described above, there is a need to better identify severity features of importance and severity score a trial for a sleep disorder severity assessment task in order to get rid of the subjective biases that metrics proposed by the medical and sleep community suffer from. Therefore, a framework is described for assessing the severity of a sleep disorder of a subject using an explainable artificial intelligence (XAI) model.

The ML model of the present disclosure relies on an interpretability-focused approach, focusing on comparing biosignal datasets to enhance the identification of severity features impacting the severity of the sleep disorder. The framework of this ML model includes a Convolutional Neural Network (CNN) for feature extraction, an Auto-Encoder (AE) for dimension reduction, a Generative Adversarial Network (GAN) for data generation, and a scoring unit for feature classification/regression. These modules can be trained sequentially using a semi-supervised curriculum learning process. The objective is to encode biosignal data provided as input into a latent feature space, aiming to maximize discriminability among a variety of trials based on biosignal datasets containing trials representing different severity of one or more sleep disorders.

The training of the ML model generates a latent space containing the most relevant information derived from the biosignal data provided as training data. This facilitates a more objective severity scoring of a trial relative to the plurality of trials contained within the biosignal dataset utilized for ML model operation. One advantage of the present ML model is its adaptability to process biosignal data from new subjects (e.g., individuals not previously included) and/or new biosignal data from the same subject (e.g., data from the same individuals collected at different time points in a longitudinal study). Another advantage lies in the ML model's ability to be trained without the need for a separate validation model or benchmark, enhancing objectivity, efficiency, and flexibility in the training process, independent of external resource availability.

The trained ML model can then be utilized to generate a severity score for biosignal data from a subject potentially affected by a sleep disorder. This severity score serves as a supportive diagnostic tool for assessing the severity of sleep-related disorders. One advantage of the generated severity score is its ability to aid in identifying the need for medical treatment in patients who might have been overlooked using standard medical scoring methods, such as AHI. Another advantage is its capacity to help mitigate the impact of medical treatment prescribed to patients who score higher on the standard medical scale. Additionally, the trained ML model can identify one or more severity features or biomarkers to assist in assessing the optimal treatment method for addressing the sleep disorder, resulting in a more personalized treatment approach for the subject. Hence, the diagnostic tool proves particularly beneficial for aiding medical practitioners during initial sleep studies conducted under time constraints. This capability helps conserve valuable resources, allowing for better allocation towards patient assistance and treatment methods.

A first overview of various aspects of the technology according to the present disclosure is given hereinbelow, after which specific embodiments will be described in more detail. This first overview is meant to aid the reader in understanding the technological concepts more quickly, but it is not meant to identify the most important or essential features thereof, nor is it meant to limit the scope of the present disclosure.

An aspect of the present disclosure relates to a system for assessing a severity of a sleep disorder of a subject, the system comprising a memory and a processor in communication with the memory, wherein the processor is configured to:
receive biosignal data associated with a plurality of trials representing different levels of severity of the sleep disorder, wherein the biosignal data includes a plurality of severity features variably impacting the severity of the sleep disorder;
train a machine learning (ML) model to encode the biosignal data from the trials into a plurality of latent vectors characterizing the severity of the sleep disorder, utilizing the severity features, and arranging the latent vectors in a latent space to increase discriminability between latent vectors representing different levels of severity;
operate the trained ML model on biosignal data obtained from the subject to generate a severity score indicative of the severity of the sleep disorder of the subject, determined from a position of the corresponding latent vector in the latent space relative to the plurality of latent vectors; and,
generate a report comprising at least the severity score for assessing the severity of the sleep disorder of the subject.

An aspect of the present disclosure relates to a computer-implemented method for assessing a severity of a sleep disorder of a subject, comprising the steps of:
receiving biosignal data associated with a plurality of trials representing different levels of severity of the sleep disorder, wherein the biosignal data includes a plurality of severity features variably impacting the severity of the sleep disorder;
training a machine learning (ML) model to encode the biosignal data from the trials into a plurality of latent vectors characterizing the severity of the sleep disorder, utilizing the severity features, and arranging the latent vectors in a latent space to increase discriminability between latent vectors representing different levels of severity;
operating the trained ML model on biosignal data obtained from the subject to generate a severity score indicative of the severity of the sleep disorder of the subject, determined from a position of the corresponding latent vector in the latent space relative to the plurality of latent vectors; and,
generating a report comprising at least the severity score for assessing the severity of the sleep disorder of the subject.

Another aspect of the present invention relates to a system for severity scoring of a plurality of severity features associated with a sleep disorder, possibly a medical sleep disorder comprising a memory and a processor in communication with the memory, wherein the processor is configured to:
receive biosignal data of a plurality of trials having a varying severity of the sleep disorder, wherein the biosignal data is acquired by one or more biosignal sensors and includes a plurality of severity features that have a varying impact on the severity of the sleep disorder;
train a ML model to learn a plurality of latent vectors corresponding to the plurality of trials, that indicate the severity of the sleep disorder, based on the plurality of severity features, and organise the plurality of latent vectors in a latent space such that discriminability between latent vectors of different severity is increased;
operate the ML model to generate a severity score that is indicative of the severity of the sleep disorder of the trial based on a position of the corresponding latent vector in the latent space relative to the plurality of latent vectors.

Another aspect of the present invention relates to a computer-implemented method for severity scoring of a plurality of severity features associated with a sleep disorder, possibly a medical sleep disorder, comprising the steps, performed by a processor, of:
receiving biosignal data of a plurality of trials having a varying severity of the sleep disorder, wherein the biosignal data is acquired by one or more biosignal sensors and includes a plurality of severity features that have a varying impact on the severity of the sleep disorder;
training a ML model to learn a plurality of latent vectors corresponding to the plurality of trials, that indicate the severity of the sleep disorder, based on the plurality of severity features, and organise the plurality of latent vectors in a latent space such that discriminability between latent vectors of different severity is increased;
operating the ML model to generate a severity score that is indicative of the severity of the sleep disorder of the trial based on a position of the corresponding latent vector in the latent space relative to the plurality of latent vectors.

Another aspect of the present invention relates to a computer program and/or a computer-readable medium comprising instructions which, when the instructions are executed by a computer comprising a processor and the processor is provided with biosignal data set of a plurality of trials having a varying severity of the sleep disorder, wherein the biosignal data is acquired by one or more biosignal sensors and includes a plurality of severity features that have a varying impact on a severity of the medical and/or sleep disorder, cause the processor to train a ML model to generate a plurality of latent vectors corresponding to the plurality of trials, that indicate the severity of the sleep disorder, based on the plurality of severity features, and organise the plurality of latent vectors in a latent space such that discriminability between latent vectors of different severity is increased, preferably maximised, and operate the ML model to generate a severity score that is indicative of the severity of the sleep disorder of the trial based on a position of the corresponding latent vector in the latent space relative to the plurality of latent vectors.

In any of the above aspect, the ML model can comprise an auto-encoder module including an encoder and a decoder, a generative adversarial network module including a discriminator, and a scoring module; wherein the auto-encoder module is configured to encode distinct features contained in the biosignal data into the plurality of latent vectors such that the biosignal data can be reconstructed; wherein the GAN module is configured to transfer the non-sparsity and generative properties from a reference distribution to the latent space through an adversarial learning process, whereby the discriminator is trained to distinguish between real data sampled from the reference distribution and fake data sampled from the latent space generated by the encoder aimed at deceiving the discriminator; and, wherein the scoring module is configured to estimate the values of the severity features based on the position of each latent vector within the latent space.

In some embodiments the AE module can be configured to extract the plurality of severity features into a latent space and to generate a reference latent space that has a generative property and a non-sparse and uniform latent space distribution.

In some embodiments the GAN module can be configured to transfer the properties of the reference latent space into the latent space by generating fake data in the latent space, and differentiating between real data sampled from the reference latent space and fake data generated in the latent space.

In some embodiments wherein the scoring module can be configured to separate the plurality of extracted severity features in the latent space based on their impact on the severity of the sleep disorder.

In some embodiments, the severity score is generated by sorting the plurality of latent vectors along on a severity direction, preferably in increasing or decreasing severity, generating a severity scale that represent the multi-dimensional direction of the latent space along the plurality of latent vectors, and assigning a severity score for the trial based on the position of the corresponding latent vector along the severity scale.

In some embodiments the ML model is configured to arrange the latent vectors in the latent space to maximise discriminability between the latent vectors representing different level of severity of the sleep disorder, preferably by assigning maximally opposite values to the latent vectors that are furthest apart in the latent space.

In some embodiments, wherein the ML model is operable to identify one or more severity features included in the biosignal data that have an impact on the severity of the sleep disorder based the plurality of latent vectors.

In some embodiments the latent space and/or reference distribution is a Gaussian distribution, preferably a multivariate Gaussian distribution.

In some embodiments, the latent space has a reconstructive property, a generative property, and a non-sparse, uniform distribution, preferably Gaussian distribution.

In some embodiments, the AE module comprises a variational auto-encoder (VAE) and/or an adversarial auto-encoder (AAE).

In some embodiments, the AE module is trained with a loss function that includes a reconstruction loss, an adversarial loss and/or a Kullback-Leibler divergence.

In some embodiments, the generator of the GAN module is trained with a loss function including a weighted sum of the AE loss and the mean of correct predictions by the discriminator and/or a binary cross-entropy (BCE) loss within a biosignal dataset, and/or the discriminator of the GAN module is trained on the difference between the mean fake predictions and the mean real predictions and/or BCE loss.

In some embodiments, the discriminator of the GAN comprises a multilayer perceptron (MLP), preferably a 3-layer MLP wherein each layer is configured to apply applies a rectifier activation function with a negative slope and batch normalization.

In some embodiments, the scoring module comprises a classifier and/or a regressor, preferably a linear classifier.

In some embodiments, the scoring module is based on a threshold transfer function, preferably comprises a single layer perceptron (SLP).

In some embodiments, an alert can be generated if the severity score exceeds a predetermined threshold.

In some embodiments the diagnostic report further comprises one or more severity features impacting the severity score of the sleep disorder of the subject.

In some embodiments one or more biomarkers indicative of the severity of the sleep disorder of the subject from the severity features can be identified and included in the diagnostic report.

In some embodiments, the biosignal data includes data from an electroencephalogram (EEG), an electrocardiogram (ECG), an electrooculogram (EOG), an electromyography (EMG), a photoplethysmogram (PPG), skin conductance, breath, heartrate, respiration airflow, and/or oxygen saturation data.

In some embodiments, the encoder and the decoder include a plurality of successive convolutional layers, wherein a number of successive convolutional layers of the encoder equals a number of successive convolutional layers of the decoder.

### DESCRIPTION OF THE FIGURES

The following description of the figures relate to specific embodiments of the disclosure which are merely exemplary in nature and not intended to limit the present teachings, their application or uses. Throughout the drawings and description, the following symbols and abbreviations are used: biosignal data 2; machine learning model 10; encoder 20; latent space 21; latent vector 22; decoder 30; reference distribution 25; discriminator 50; fake data 51; real data 52; scoring module 70; severity scale 71; severity score 72.

FIG. 1 shows a schematic representation of a machine learning (ML) model architecture 10 comprising an adversarial auto-encoder (AAE) in accordance with aspects of the present invention.

FIG. 2 shows a schematic representation of a ML model 10 comprising a variational auto-encoder (VAE) in accordance with other aspects of the present invention.

FIGS. 3A to 3C show a 2D representations of the encoder latent space using t-distributed stochastic neighbour embedding (t-SNE) transform. Each sample represents one of the 6992 OSA trials. Details of the figures are discussed in Example 1 of the present disclosure.

In particular, FIG. 3A shows the encoder latent space 21 with the training phase of an AE module completed.

In particular, FIG. 3B shows the encoder latent space 21 with the training phase of a GAN module completed.

In particular, FIG. 3C shows the encoder latent space 21 with the training phase of a scoring module 70 completed. The severity scale 71 represents the severity direction obtained by sorting the trials based on their severity score.

FIGS. 4A to 4D show the biomarkers identification performed by comparing the power signal, by channel, of the OSA trials sorted by severity score (E [0,1]) along the severity direction obtained by sorting the latent vectors. Details of the figures are discussed in Example 1 of the present disclosure.

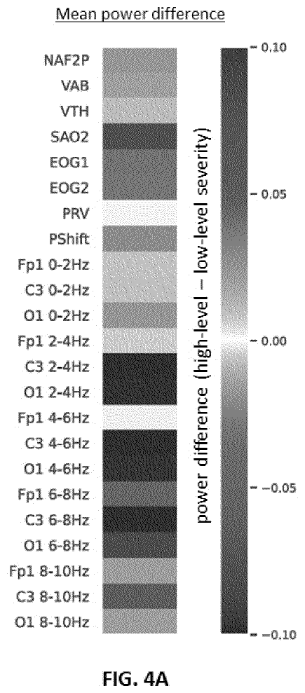

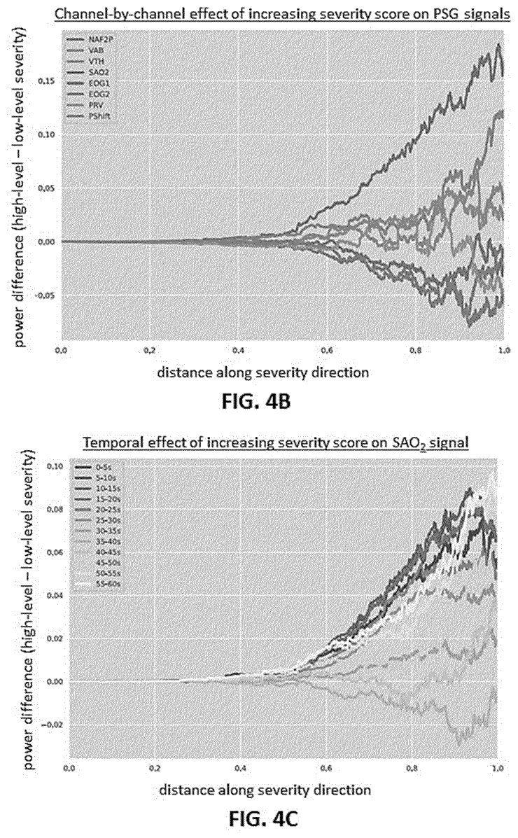

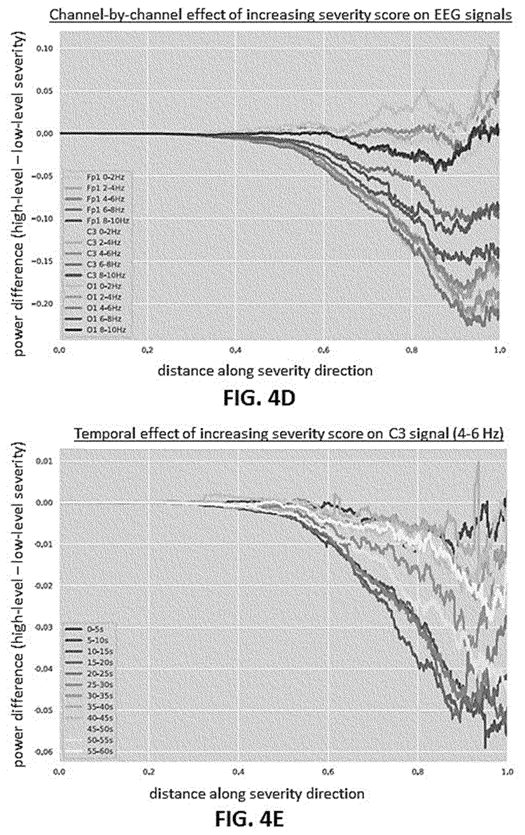

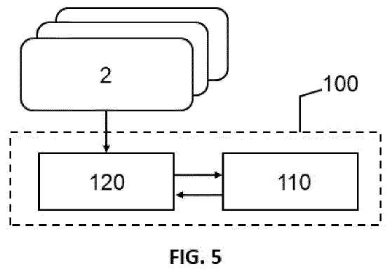

In particular, FIG. 4A shows the Mean power difference across OSA trials obtained by subtracting, for each channel separately, the power signal of each trial from the power signal of trials of higher severity scores.

In particular, FIG. 4B shows the Channel-by-channel mean power difference of PSG channels excluding EEG channels. The x-axis represents the distance, along the severity direction, between the trials being compared. A distance of 0 means a trial is compared to itself, a distance of 1 means the comparison between the trial of lowest severity score and the trial of highest severity score.

In particular, FIG. 4C shows the Time window-by-time window mean power difference of the SAO2 channel (channel of highest absolute mean power difference).

In particular, FIG. 4D shows the Channel-by-channel mean power difference of EEG channels.

In particular, FIG. 4E shows the Time window-by-time window mean power difference of the C3 channel on the 4-6 Hz frequency band (channel of highest absolute mean power difference).

FIG. 5 shows an implementation of the ML model 10 on a computing system 100 in accordance with aspects of the present invention.

### DETAILED DESCRIPTION

In the following detailed description, the technology underlying the present invention will be described by means of different aspects thereof. It will be readily understood that the aspects of the present disclosure, as generally described herein, and illustrated in the figures, can be arranged, substituted, combined, and designed in a wide variety of different configurations, all of which are explicitly contemplated and make part of this disclosure. This description is meant to aid the reader in understanding the technological concepts more easily, but it is not meant to limit the scope of the present invention, which is limited only by the claims.

The present invention relates to a machine learning (ML) configured for severity scoring of a trial or a segment of a trial that is associated with a sleep disorder, possibly a medical sleep disorder. The present invention is described by way of example in terms of severity scoring of a trial associated with a sleep disorder, it can be appreciated that, at least in principle, any biosignal data comprising a plurality of severity features that have a varying impact on the severity of an associated sleep disorder can benefit from the present ML model. Therefore, the present ML model can be regarded as adaptive scoring model in the sense that it can be readily adapted for severity scoring of a trial based on a plurality of associated severity features present in biosignal data comprising a plurality of trials of the associated sleep disorder. The ML model of the present invention relies on an interpretability-focused approach based on the comparison between trials or samples included in biosignal dataset to maximize the discriminability between severity features impacting the severity of the sleep disorder.

The framework of the herein described ML model comprises a Convolutional Neural Network (CNN) for feature extraction, an Auto-Encoder (AE) module for dimension reduction, a Generative Adversarial Network (GAN) module for imposing latent space distribution, and a scoring module for classification and/or regression. The modules can be trained sequentially using a semi-supervised curriculum learning process with the objective of encoding biosignal data that is provided as input into a latent feature space to maximise the discriminability between biosignal datasets of different sleep disorder severity. In this way, the framework provides a latent space that contains the majority of the information contained in the biosignal data, is usable with new biosignal datasets (for example, biosignal data of a new subject), and/or allows a fair comparison between different biosignal datasets (for example, datasets acquired at different points in time).

As used herein “autoencoder” (AE) refers to a neural network architecture that learns an encoding function that transforms the biosignal data provided as input, and a decoding function that recreates the biosignal data from the encoded representation. The autoencoder can learn an efficient representation (encoding) for dimensionality reduction. Specific embodiments of the AE will be described later.

As used herein “Generative Adversarial Network” (GAN) refers to neural network architecture implemented with two differentiable networks, a generator and discriminator. The generator (confounded with the encoder of the AE) is configured to generate data that appears to be drawn from the desired distribution. The discriminator is then configured to assign a (high) value to real (nongenerated) data and another (low) value to fake (generated) data. For example, a value of “1” can be assigned to real data and “0” to fake data. Specific embodiments of the GAN will be described later.

As used herein, “subject” refers to a human person or animal. The subject may suffer from one or more (medical or nonmedical) disorders that affect their health and wellbeing. The subject may suffer from one or more sleep disorders that affect the quality of the subject's sleep. For example, the subject may be a patient receiving care in a clinical environment or a volunteer in a research study. The subject may be a healthy person without a relevant sleep disorder but suffer from one or more sleep disturbances.

As used herein, “biosignal” refers to a recording of a biological event in a subject that can be measured and monitored. The event may, for example, include a beating heart or respiratory airflow. The electrical, chemical, and mechanical activity that occurs during this biological event produces signals that can be measured with a sensor device, herein referred to as “biosignal sensor”. The biosignal sensor can, for example, include a biomedical or medical sensor that is attachable or wearable on the body of the subject, or a sensor that monitors the subject from a distance. The biosignal data can, for example, within the context of sleep disorder research, include data from an electroencephalogram (EEG), an electrocardiogram (ECG), an electrooculogram (EOG), electrooculogram (EOG), electromyography (EMG), a photoplethysmogram (PPG), skin conductance, breath, heartrate, respiration airflow, oxygen saturation data, and so on; the provided list is non-exhaustive and exemplary in nature only. Each set of biosignal data includes one or more “features” that can be used as variables for classification. For example, the features present in ECG data can include the wave type, the time span, the amplitude and the slope of the waveform. The biosignal data may be acquired in a clinical or research environment or at home. The biosignal data can be used as input to the herein described system.

As used herein, a “trial” refers to a segment of data that is provided as input to the machine learning model for training purposes. The data corresponding to the trial may include biosignal data of one or more subjects acquired by one or more biosignal sensors that may be relevant to the same sleep disorder or a plurality of different sleep disorders. In some embodiments, this data may be acquired as part of a “study” of a (medical or nonmedical) sleep disorder than can affect the health and wellbeing of the monitored subjects. The study may be designed to study one or more sleep disorders and/or one or more sleep disturbances. For example, the trial may be a clinical trial of a subject receiving care in a clinical environment or a research trial of a subject volunteer in a research study. The biosignal data may advantageously be acquired at the same time or within a relevant time period. The study may be split up into different segments, for example, based on sleeping time or focusing on specific (sleep) stages, such as REM or nonREM.

As used herein, “severity feature” refers to a feature in the biosignal data of a trial as defined above, which has an impact on the severity of a sleep disorder, possibly a medical sleep disorder at least to an extent; the severity feature is relevant for the assessment of how and to which degree the sleep disorder and/or medical sleep disorder may affect the health and wellbeing of the subject. The severity feature may advantageously have a (clinically) significant and noticeable impact on the severity of a sleep disorder and/or medical sleep disorder to allow for a more reliable scoring. The impact of the severity feature can be dependent on the type of sleep disorder and on one or more subject specific parameters, such as age, sex, weight, and so on. The nature and impact of the severity feature can be determined empirically by healthcare professional, such as a clinician, sleep researcher or trained technician.

As used herein, “biomarker” refers to a relevant part of the biosignal data associated with a sleep disorder, possibly a medical sleep disorder that may serve as an objective indicator of the presence or severity of the sleep disorder and/or medical sleep disorder. Specifically, although biosignal data may include a large number of features, at least part of these features may be not relevant or may be not relevant for a type of sleep disorder and/or medical sleep disorder. For example, within the context of a sleep disorder, a biomarker could be a particular pattern of brain activity during specific stages of sleep, abnormalities in breathing or hear rate patterns detected during sleep, and so on. The biosignal data may include one or more biomarkers that may be related or unrelated to each other.

As used herein, a “latent vector” is known in the art as a high-dimensional vector representing an encoding of information learned by the machine learning model described herein. The latent vector encompasses the variables describing the features contained in the training data provided as input from one or more trials as defined above. Advantageously, the number of trials may be represented by a corresponding number of latent vectors.

As used herein, a “latent space” refers to a multi-dimensional space formed by the latent vectors encoded by the trained machine learning model described herein. The latent space represents the abstract feature space where data points are embedded in a compressed form. Each dimension in this space corresponds to a latent variable of a feature learned by the model. The arrangement of latent vectors within the latent space captures the underlying structure of the input data, facilitating various transformations, interpolations, and generative processes. The latent space is often characterized by its dimensionality, distribution, and the relationships between latent vectors, which are learned during the training of the machine learning model. Accordingly, the latent space encompasses the plurality of latent vectors corresponding to the plurality of trials included in the provided training data.

As used herein, “latent space properties” refers to characteristics inherent in the high-dimensional vectors representing information encoded by the machine learning model described herein. Latent vectors possess dimensionality corresponding to the number of latent features learned by the model, adhering to specific distributions that influences their role in generative processes and data transformations. For example, the latent space properties may include a reconstructive property, a generative property and/or a non-sparse, uniform distribution, such as a Gaussian distribution. In this way, the latent space can allow an efficient comparison between trials of varying severity of the sleep disorder and/extending the comparison to new data, for example, biosignal data of a new subject that was not included in the biosignal data for training the machine learning model and/or biosignal data of the same subject but at a new time, for example, as part of a longitudinal study.

As used herein, “severity score” refers to a score assigned to a trial as described herein and/or a sleep disorder that is representative of the (medical or nonmedical) impact of the trial and/or sleep disorder on the health and wellbeing of the subject, that is, the “severity” of the disorder. For example, the severity score of a sleep disorder can be based on the number of disturbances occurring overnight. Typically, the severity is based on the total impact of a plurality of severity features. The severity score therefore aims to assign an objective score to a particular (single) biosignal data of a subject relative to the plurality of trials that are provided as input to the trained machine learning model. The severity score can include a numerical value, such as an integer, ranging from a predetermined minimum value representing the lowest severity to a predetermined maximum value representing the highest severity of the associated sleep disorder.

In some embodiments, the severity score may be determined through the use of a “severity scale” that represent the multi-dimensional direction of a latent space including the plurality of latent vectors having a varying severity of the sleep disorder. In this way, the severity score reflects the overall severity of a particular trial based on the position of the corresponding latent vector on the severity scale within the latent space.

An overview of various aspects of the technology of the present invention is given hereinbelow, after which specific embodiments will be described in more detail. This overview is meant to aid the reader in understanding the technological concepts more quickly, but it is not meant to identify the most important or essential features thereof, nor is it meant to limit the scope of the present disclosure. When describing specific embodiments, reference is made to the accompanying drawings, which are provided solely to aid in the understanding of the described embodiment.

An aspect of the present invention relates to a system comprising a memory and a processor in communication with the memory,
wherein the memory is configured to receive biosignal data associated with a plurality of trials representing different levels of severity of the sleep disorder, wherein the biosignal data includes a plurality of severity features variably impacting the severity of the sleep disorder; and wherein the processor is configured to:
   train a machine learning model to encode the biosignal data from the trials into a plurality of latent vectors characterizing the severity of the sleep disorder, utilizing the severity features, and arranging the latent vectors in a latent space to increase discriminability between latent vectors representing different levels of severity;
   operate the trained machine learning model on biosignal data obtained from the subject to generate a severity score indicative of the severity of the sleep disorder of the subject, determined from a position of the corresponding latent vector in the latent space relative to the plurality of latent vectors; and,
   generate a report comprising at least the severity score for assessing the severity of the sleep disorder of the subject.

Another aspect of the present invention relates to a computer-implemented method comprising the steps, preferably performed by a processor, of:
receiving biosignal data associated with a plurality of trials representing different levels of severity of the sleep disorder, wherein the biosignal data includes a plurality of severity features variably impacting the severity of the sleep disorder;
training a machine learning model to encode the biosignal data from the trials into a plurality of latent vectors characterizing the severity of the sleep disorder, utilizing the severity features, and arranging the latent vectors in a latent space to increase discriminability between latent vectors representing different levels of severity;
operating the trained machine learning model on biosignal data obtained from the subject to generate a severity score indicative of the severity of the sleep disorder of the subject, determined from a position of the corresponding latent vector in the latent space relative to the plurality of latent vectors; and,
generating a report comprising at least the severity score for assessing the severity of the sleep disorder of the subject.

Another aspect of the present disclosure relates to a computer program and/or a computer-readable medium comprising instructions which, when the instructions are executed by a computer comprising a processor and the processor is provided with biosignal data associated with a plurality of trials representing different levels of severity of the sleep disorder, wherein the biosignal data includes a plurality of severity features variably impacting the severity of the sleep disorder, cause the processor to train a machine learning model to encode the biosignal data from the trials into a plurality of latent vectors characterizing the severity of the sleep disorder, utilizing the severity features, and arranging the latent vectors in a latent space to increase discriminability between latent vectors representing different levels of severity; and operate the trained machine learning model on biosignal data obtained from the subject to generate a severity score indicative of the severity of the sleep disorder of the subject, determined from a position of the corresponding latent vector in the latent space relative to the plurality of latent vectors; and, generate a report comprising at least the severity score for assessing the severity of the sleep disorder of the subject.

In any of the above aspect, the machine learning model (ML) may comprises an auto-encoder module including an encoder and a decoder, a generative adversarial network module including a discriminator, and a scoring module; wherein the auto-encoder module is configured to encode distinct features contained in the biosignal data into the plurality of latent vectors such that the biosignal data can be reconstructed; wherein the GAN module is configured to transfer the non-sparsity and generative properties from a reference distribution to the latent space through an adversarial learning process, whereby the discriminator is trained to distinguish between real data sampled from the reference distribution and fake data sampled from the latent space generated by the encoder aimed at deceiving the discriminator; and, wherein the scoring module is configured to estimate the values of the severity features based on the position of each latent vector within the latent space.

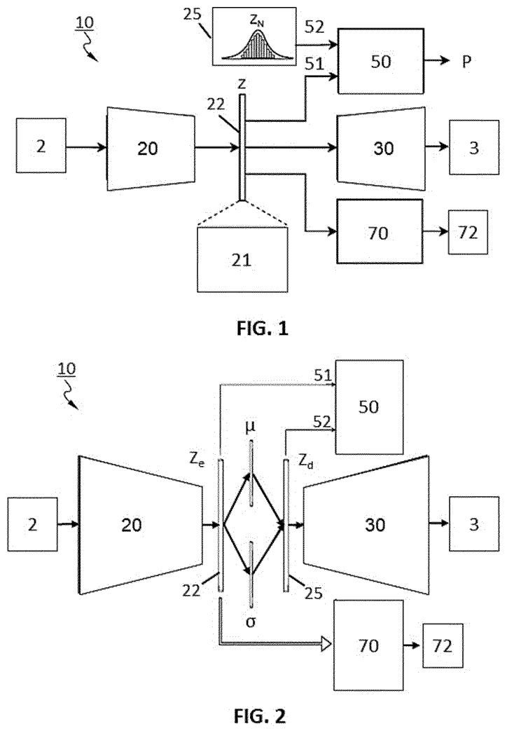

The present system and embodiments thereof will be discussed further with reference to FIG. 1, which is a block diagram illustrating the architecture of the ML model 10 in accordance with aspects of the present invention. It is understood that additional components or modules can be included.

FIG. 1 illustrates the ML model 10 as comprising an autoencoder (AE) module (illustrated centrally) that comprises an encoder an encoder 20 and a decoder 30. Each of the encoder 20 and the decoder 30 can comprise a plurality of convolutional layers, that is, a first succession of convolutional layers for the encoder 20 and a second succession of convolutional layers for the decoder 30. The input biosignal data is provided to the encoder 20. The output of the encoder is the latent vector z, which is connected to the decoder 30 to output a reconstructed version of the input biosignal 3. The whole structure represents the AE module.

The successive convolutional layers of the encoder 20 are configured to sequentially encode biosignal data provided as input 2 to the AE into a latent space embedding, referred to herein as the “encoder latent space” 21. The encoder latent vector 22 can form the last layer of the plurality of convolutional layers of the encoder 20. In this way, biosignal data provided as input 2 to the AE can undergo different transformations across the convolutional layers of the encoder 20 that reduce the dimensionality of the data, more specifically, by generating a reduced number of computed features based on the original features present in the biosignal data; this process is referred to as feature extraction. The encoder latent vector 22 can be considered as a projection of the biosignal input data into an N-dimensional space, and thus includes a representation of extracted feature in the latent space that includes the features in the biosignal input data. The purpose of these computed features is to be maximally discriminative for the categories that have to be classified, as will be discussed later.

As described further below, in some embodiments the AE module can be configured as a variational auto-encoder (VAE) and/or an adversarial auto-encoder (AAE). The use of VAE instead of conventional AE enables to estimate the distribution (characterized by mean and standard deviation) of the latent space and to study the relationship between their distribution instead of creating a one-to-one relationship between latent vectors. In some embodiments the AE can be trained with a loss function, preferably a loss function that includes a reconstruction loss and/or Kullback-Leibler divergence. The latter embodiment is preferential for training a variational auto-encoder. In some other embodiments the AE can be trained with a loss function, preferably a loss function that includes an adversarial loss. The latter embodiment is preferential for training an adversarial auto-encoder. Accordingly, the AE can be configured to generate a reference latent space that is assigned specific latent space properties.

In FIG. 1, an embodiment of the AE module is presented featuring an adversarial auto-encoder (AAE). Within this configuration, the auto-encoder module AE can undergo training utilizing a reconstruction loss, which evaluates the fidelity of reconstructing input data from the latent space. Further, the GAN module facilitates the transfer of characteristics from a predefined prior distribution 25 to the latent space 21. The ML model can be trained through a combined approach involving both an auto-encoder loss (reconstruction loss) and a GAN loss (adversarial loss). The GAN loss works to align the distribution of the latent space with the predefined prior distribution by simultaneously training a discriminator network. This network distinguishes between samples directly drawn from the desired reference prior distribution and samples generated by the encoder, which attempts to deceive the discriminator.

In an embodiment the AE can be configured to generate another latent vector that forms the input of the plurality of convolutional layers of the decoder 30, referred to herein as the “decoder latent vector”. In the ML model of FIG. 2, the decoder latent space can obtain specific latent space properties through the auto-encoding training process of the AE, as will be described later. These properties may preferably include a generative property and a non-sparse and uniform latent space distribution. For example, in an embodiment where the AE is a variational auto-encoder, the decoder latent vector may be generated by estimating the mean (μ) and the standard deviation (σ) of the Encoder latent space obtained by gathering the different encoder latent vectors 22 generated from the dataset using dense layers—although other methods are possible to obtain similar space properties. In another embodiment, only a single latent vector may be generated—for example, a ML model comprising an adversarial auto-encoder comprising one latent vector may be sufficient.

The successive convolutional layers of the decoder 30 can be configured to sequentially decode data from the first convolutional layer of the plurality of convolutional layers of the decoder 30, that is, from the decoder latent vector, to derive a reconstructed version of the biosignal data provided as input 2 as an output 3 of the ML model 10. In some embodiments, the decoder can be the mirrored version of the encoder where the convolutions preferably can be replaced by transposed convolutions and the max pooling layers preferably can be replaced by max unpooling ones. In some embodiments, the number of successive convolutional layers of the encoder can equal a number of successive convolutional layers of the decoder.

In some embodiments the reference latent space, possibly decoder latent vector, can have a multivariate Gaussian distribution. Although any distribution resulting in a non-sparse, uniform latent space can be considered, the Gaussian distribution is the simplest one and provides a good outcome; in this way, the complexity of the model can be reduced. Gaussian distribution allows for a fair comparison between biosignal datasets by performing a directional study within the feature space, that is, by looking for the direction responsible for severity encoding.

In FIG. 2, an embodiment of the AE module is presented featuring a variational auto-encoder (VAE). Within this configuration, the auto-encoder module AE can be configured to impose a desired distribution for the latent representation of the provided input dataset. It does this by employing the generative adversarial network (GAN) described further below to match the distribution of the latent space to the distribution of the decoder latent space. The ML model can be trained through a combination of an auto-encoder loss (reconstruction loss) and an additional loss component specific to the VAE. This loss component aims to regularize the learned latent space distribution, encouraging it to adhere to a predefined prior distribution. Through this training process, the VAE aims to generate latent representations that accurately capture the underlying structure of the input data while also conforming to the specified prior distribution.

Compared to the previously discussed embodiment of FIG. 1, the latent vector z is essentially replaced by an encoder latent vector 22 connected to a decoder latent vector 32 using 2 distinct linear layers representing the mean (u) and the standard deviation (o) of the encoder latent vector. This results in the AE becoming a VAE instead. Consequently, the difference between VAE and AAE is based on how the distribution of the latent space is imposed (ensemble of “z” vectors obtained from the entire dataset), which can be Gaussian for example.

In the VAE of FIG. 2, the distribution can be obtained by designing the encoder latent vector (Ze) to be as similar as possible to the decoder latent vector (Zd) through adversarial training (GAN) discussed further below. The decoder latent vector Zd can be configured to be Gaussian, as it results from the combination of the “μ” and “σ” layers: Zd=μ+ε*σ, where ε is a random variable. In comparison, in the AAE of FIG. 1, Z is imposed to be as similar as possible to a chosen distribution Z_{N}, for example Gaussian. Alternatively, Z_{N} can be external from the architecture and only use for comparative purpose. It should therefore be understood that any further discussed embodiments of the ML model comprising VAE, as discussed with reference to FIG. 2, also constitute embodiments of the ML model comprising AAE, as discussed with reference to FIG. 1, unless otherwise specified. Although some adaptations may be necessary, these fall within the capabilities of a skilled person.

Continuing with FIG. 1, the next module of the ML model 10 (illustrated in the top-right) is a GAN module, combining the encoder 20 and the discriminator 50, such that is configured for transferring the properties of the reference latent space, possibly decoder latent vector, into the latent space of the encoder latent vector 22. IN an embodiment, the GAN can include a generator (confounded with the encoder 20) that generates fake data 51 that advantageously resembles the real (sampled from the reference latent space) data 52 and a discriminator 50 that discriminates between the fake (generated) data 51 and real (sampled from the reference latent space) data 52. In the present embodiment, the GAN is configured to force the encoder 20 to act as a generator and generate fake data 51 sampled from the encoder latent space 21.

In some embodiments the generator of the GAN module can be trained with a loss function in which the generator is a weighted sum of the AE loss and the mean of correct predictions by the discriminator 50 within a biosignal dataset and/or a binary cross-entropy (BCE) loss.

In some embodiments the discriminator 50 of the GAN module can be trained with a loss function based on the difference between the mean fake predictions and the mean real predictions and/or a binary cross-entropy (BCE) loss. For example, the discriminator's predictions of generated latent vector and the value representing the fake (generated) data, which can set to 0, and the discriminator's predictions of reference Gaussian samples and the value representing the real (sampled from the reference latent space) data, which can be set to 1.

In some embodiment the discriminator 50 of the GAN module can comprise a multilayer perceptron (MLP), preferably a 3-layer MLP wherein each layer is configured to apply a rectifier activation function with a negative slope and batch normalization. The output activation function can be a sigmoid function. The AE can bring reconstructive property to the encoder latent vector 22 while the GAN transfers the generative and distribution properties from the reference distribution to the encoder latent vector 22. The reconstruction ability ensures a direct relationship between the feature space and all the independent input channels, allowing users to evaluate the relevance of the proposed classification. The generative ability ensures the model to be usable for new input data, for example based on a new subject, by avoiding part of the space not to be characterized. The non-sparce, uniform distribution allows for a fair comparison between a plurality of biosignal datasets trials by performing a directional study within the feature space.

The next module of the ML model 10 of FIG. 1 (illustrated in the bottom-right) is a scoring module 70 configured to separate the plurality of extracted features that have a different impact on the severity of the sleep disorder in the latent space of interest. This in turn, allows for allocating a specific weight to the plurality of extracted features to make a classification. Preferably, the feature space is manipulated to obtain a latent space that has the above latent space properties, including a reconstructive property, a generative property, and a non-sparse, uniform distribution.

In some embodiments, the scoring module can comprise a single layer perceptron (SLP). The output activation function can be a soft-max function.

In some embodiments the severity score can be defined as discrete levels and/or as continuous scale value. The plurality of generated latent vectors having a different severity score can be organised based on their position which determines the value of their severity score.

In some embodiments the discriminability between the latent vectors representing different severity can be maximised by assigning maximally opposite values to the latent vectors that are furthest apart in the latent space. This can be achieved by assigning distinct severity scores to these latent vectors, such as assigning scores of 0 and 1 as examples of maximally opposite values. In this way, a severity scale can be defined between the assigned severity scores, for example, ranging from 0 to 1.

In some embodiments, the ML model may be initially trained using training data, although parameters underlying and/or used by the ML model may also be occasionally or continually refined based on later interactions and/or collaborations with a user of the below described system. As the performance of the scoring module depends on its ability to sort the features in the biosignal data from the least severe to the most severe, different validations can be considered. For example, a metric can be implemented to exclude one or more datasets related to one or more subjects in a dataset comprising a plurality of subjects. This allows for validating the model towards new subjects based on the trained model. Hence, no physical model or benchmark is required to perform a validation.

After training the ML model as described above, inference process can be employed to process ‘new’ biosignal data from a new subject (e.g., individuals not previously included in the training data) and/or from the same subject (e.g., data from individuals previously included in the training data but at different time points in a longitudinal study). Specifically, operating the trained ML model on biosignal data obtained from a subject potentially affected by a sleep disorder can be used to generate a severity score indicative of the severity of the sleep disorder. This score is based on the position of the corresponding latent vector in the latent space relative to the plurality of latent vectors.

Subsequent to the inference step, diagnostic information can be collected to generate a report as output. In one embodiment, the diagnostic information in the report can include at least the severity score for assessing the severity of the sleep disorder. Various methods can be employed to present the severity score in the report. For instance, a binary scoring system may be utilized by comparing the severity score to a predetermined threshold, with the report indicating ‘no treatment necessary’ or ‘treatment necessary’. Alternatively, a grading scale may be implemented, assigning specific values to the severity score, with the report indicating ‘no treatment necessary’, ‘minimal or non-invasive treatment advised’, or ‘maximal or device-assisted treatments advised’. The customization of report parameters is adaptable to the user's requirements.

Furthermore, the diagnostic report can include one or more severity features that impact the severity score of the sleep disorder of the subject. Additionally, the method or system may involve identifying one or more biomarkers indicative of the severity of the sleep disorder of the subject from these severity features. Consequently, the diagnostic report may also include these identified biomarkers of the subject. In some embodiments the diagnostic report may further comprises one or more severity features impacting the severity score of the sleep disorder of the subject. In the context of the herein described method, the severity features refer to specific measurable aspects or characteristics of the sleep disorder that contribute to assessing its severity. These features could include physiological parameters like heart rate variability, respiratory patterns, brainwave activity, or other relevant biosignal data collected from the biosignal sensors.

In some embodiments the diagnostic report can further comprise one or more biomarkers indicative of the severity of the sleep disorder of the subject from the severity features. In the context of the herein described method the biomarker refers to a specific type of severity feature that serves as an objective indicator of the presence or severity of a disease or condition. In the context of a sleep disorder, a biomarker could be a particular pattern of brain activity during specific stages of sleep, abnormalities in breathing or hear rate patterns detected during sleep, and so on. These biomarkers are intended to provide guidance and support to the medical practitioner in determining an optimal treatment method if deemed necessary.

For instance, the report may include an overview delineating the various respiratory events encountered by the subject, along with their associated severity scores, timing, and key biomarkers responsible for severity. In one embodiment, the biomarkers may include:
NAF2P: duration of respiratory events;
SAO₂: hypoxic burden, including desaturation of at least 4% and time spent below 90% saturation;
PRV: standard deviation of RR values (SDNN), root mean square of successive RR differences (RMSSD), and entropy; and/or
Phase shift: average and maximum shift of thoracic vs. abdominal belts movements during respiratory events. It should be noted that the report is customizable to incorporate various biomarkers depending on the type and severity of the sleep disorder.

The inclusion of one or more severity features and/or one or more biomarkers in the diagnostic report may enable a medical professional to more effectively evaluate the appropriate treatment approach for addressing the specific sleep disorder. This inclusion also offers insights into underlying patterns of the sleep disorder, potentially overlooked by medical professionals operating under time constraints. In some embodiments the diagnostic report may further comprise treatment recommendations based on the relevant diagnostic information, such as the severity score, one or more severity features, and/or one or more biomarkers as described above. These recommendations may be determined by correlating the diagnostic information with data stored in a remote resource, such as a library containing diverse treatment options. For instance, the treatment recommendations may encompass options such as ‘lifestyle changes’, ‘medication-assisted treatment’, ‘device-assisted treatment’, or a combination thereof. The customization of treatments is adaptable to the user's requirement. These recommendations are intended to provide guidance and support to the medical practitioner in determining an optimal treatment method if deemed necessary.

Another aspect of the present invention relates to a method of treating a sleep disorder of a subject, such as sleep apnea, comprising the steps of:
acquiring subject biosignal data acquired by one or more biosignal sensors;
annotating the acquired biosignal data for one or more respiratory events;
applying the computer-implemented method as described herein on the acquired biosignal to generate a severity score that is indicative of the severity of one or more respiratory events, preferably for each of the respiratory events;
evaluating the severity score for one or more respiratory events relative to a predetermined threshold, preferably for each of the respiratory events, to determine a medical treatment to treat the sleep disorder.

Another aspect of the present invention relates to a method of treating a sleep disorder of a subject, such as sleep apnea, comprising the steps of:
acquiring subject biosignal data acquired by one or more biosignal sensors;
annotating the acquired biosignal data for one or more respiratory events and calculating the Apnea-Hypopnea Index (AHI) from the one or more respiratory events;
applying the computer-implemented method as described herein on the acquired biosignal to generate a severity score that is indicative of the severity of one or more respiratory events, preferably for each of the respiratory events;
comparing the AHI with the generated severity score to identify a discrepancy relative to a predetermined threshold, and adjusting the treatment options for the subject if any discrepancy is identified.

In an exemplary embodiment, the medical treatment for patients afflicted with obstructive sleep apnea can be classified into four tiers: (1) No treatment, (2) Mild treatment, which encompasses lifestyle adjustments such as weight management, increased physical activity, and smoking cessation encouragement; (3) Moderate treatment, involving methods like positional therapy (e.g., belts), medication (e.g., wakefulness-promoting agents like Provigil and Nuvigil, or sleep aids such as benzodiazepines or barbiturates); and (4) Intensive treatment, including the use of Continuous Positive Airway Pressure (CPAP) or mandibular advancement devices. It should be noted that additional treatment modalities may exist, and the provided list is not exhaustive.

Traditionally, treatment selection relies on parameters such as the Apnea-Hypopnea Index (AHI), comorbidities (e.g., hypertension, diabetes, cardiac or renal insufficiency, hypothyroidism, obesity, stroke), and symptoms (e.g., excessive daytime sleepiness, snoring, morning headaches, dry mouth, sore throat, attention disturbances). However, current practices often underutilize the wealth of information contained in Polysomnography (PSG) signals when making treatment decisions, resulting in some patients receiving inadequate treatment. The severity score described herein has the potential to bridge this gap by offering valuable insights derived directly from PSG signal morphology when implemented in the treatment method as described above.

With reference to FIG. 5, an implementation of the herein described ML model 10 is described on an exemplary computer system 100. The system 100 can include a processor 110 and a memory 120 configured to be in communication with each other. The processor 110 and the memory 120 can be components of a computer system, where the computer device can be, for example, a desktop computer, a laptop computer, a server, and/or other types of computer devices. In some examples, the processor 110 can be one or more cores among a multi-core processor, a special purpose processor, a programmable device, and/or other types of hardware. The memory 120 is configured to receive and store one or more biosignal data sets 2 as input and communicate the stored biosignal data to the processor 110.

The computer system may be described in the general context of computer system executable instructions, such as program modules, being executed by a computer system. Generally, program modules may include routines, programs, objects, components, logic, data structures, and so on that perform particular tasks or implement particular abstract data types. The computer system may be practiced in distributed cloud computing environments where tasks are performed by remote processing devices that are linked through a communications network. In a distributed cloud computing environment, program modules may be located in both local and remote computer system storage media including memory storage devices.

System memory 120 can include computer system readable media in the form of volatile memory, such as random-access memory (RAM) and/or cache memory or others. Computer system may further include other removable/non-removable, volatile/non-volatile computer system storage media. By way of example only, storage system 18 can be provided for reading from and writing to a non-removable, non-volatile magnetic media (e.g., a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 14 by one or more data media interfaces.

In some embodiments the memory can store a training set of biosignal data. In some embodiments, memory can store biosignal data that has been received from a biosignal sensor device. For example, within the context of sleep disorder research, the biosignal sensor device may include a device configured for measuring an electroencephalogram (EEG), an electrocardiogram (ECG), an electrooculogram (EOG), an electromyography (EMG), a photoplethysmogram (PPG), a skin conductance, a breath, a heartrate, a respiration airflow, an oxygen saturation data, and so on. In some embodiments, the memory can be replaced with one or more sensors measuring a biosignal from a subject.

The system 100 can further include a model builder, one or more application programming interface(s) (API), one or more data model repositories, and one or more data management systems. In some examples, the model builder can include code, such as source code, object code, and/or executable code. The processor 110 can be configured to implement the model builder to build and train a ML model 10 according to the present invention using various learning techniques, such as deep learning techniques. Specific training and structure of the ML model 10 are described in more detail in other parts of the present description.

In some examples, one or more components of the system 100 can be components of a cloud computing platform. In some examples, the model builder can deploy the trained ML model to be run by the processor 110. In some examples, the model builder or the processor 110 can deploy or distribute the trained model 10 to one or more devices or processors outside of the system 100, such that the one or more devices or processors can run the trained model to receive individual inputs and generate severity scores for new biosignal training datasets and/or generate predictions for an individual subject based on individual input.

The data management system can be configured to access data stored in the data model repositories. The data management systems can be operated by respective end users. For example, an end user may provide biosignal data as input to the system 100 and an end user may receive an output generated by the system 100. The system 100 can provide a platform for an end user to generate and interact with the data prior and after processing by the ML model 10, for example, to determine specific severity scores for specific severity features. For example, an end user can select and/or upload one or more biosignal datasets, and the processor 100 can apply or run the ML model 10 to generate various severity scores for the selected or uploaded biosignal datasets.

To this end, the computer system be configured to communicate with one or more external devices such as a keyboard, a pointing device, a display, etc.; one or more devices that enable a user to interact with computer system; and/or any devices (e.g., network card, modem, etc.) that enable computer system to communicate with one or more other computing devices. Such communication can occur via Input/Output (I/O) interfaces. Still yet, computer system can communicate with one or more networks 24 such as a local area network (LAN), a general wide area network (WAN), and/or a public network (e.g., the Internet) via network adapter.

The methods described herein may be implemented in hardware, software, firmware, or any combination thereof. If implemented in software, the functions may be stored, as one or more instructions or code, on and/or transmitted over a computer-readable medium and executed by a hardware-based processing unit. Computer-readable media may include computer-readable storage media, which corresponds to a tangible medium such as data storage media, or communication media including any medium that facilitates transfer of a computer program from one place to another (e.g., pursuant to a communication protocol). In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fibre-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibres, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

As used herein, the terms “one embodiment” or “an embodiment” means that a particular feature, structure or characteristic described in connection with the embodiment is included in at least one embodiment of the present disclosure. Thus, appearances of the phrases “in some embodiments” in various places throughout this specification are not necessarily all referring to the same embodiments.

As used herein, the terms “comprising”, “comprises” and “comprised of” are synonymous with “including”, “includes” or “containing”, “contains”, and are inclusive or open-ended and do not exclude additional, non-recited members, elements or method steps. The terms “comprising”, “comprises” and “comprised of” when referring to recited members, elements or method steps also include embodiments which “consist of” said recited members, elements or method steps. The singular forms “a”, “an”, and “the” include both singular and plural referents unless the context clearly dictates otherwise.

As used herein, the terms “connected” or “coupled” reflect a functional relationship between the described objects or devices, that is, the terms indicate the described objects must be connected in a way to perform a designated function which may include a direct or indirect connection in an electrical or nonelectrical (i.e. physical) manner, as appropriate for the context in which the term is used.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

As used herein, the term “about” is used to provide flexibility to a numerical value or range endpoint by providing that a given value may be “a little above” or “a little below” said value or endpoint, depending on the specific context. Unless otherwise stated, use of the term “about” in accordance with a specific number or numerical range should also be understood to provide support for such numerical terms or range without the term “about”. For example, the recitation of “about 30” should be construed as not only providing support for values a little above and a little below 30, but also for the actual numerical value of 30 as well.

The recitation of numerical ranges by endpoints includes all numbers and fractions subsumed within the respective ranges, as well as the recited endpoints. Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the disclosure described herein are capable of operation in other sequences than described or illustrated herein.

As used herein, the term “improved” with reference to the performance of devices or objects is a measure of a benefit obtained based on a comparison to similar devices or objects in the prior art. Furthermore, it is to be understood that the degree of improved performance may vary between disclosed embodiments and that no equality or consistency in the amount, degree, or realization of improved performance is to be assumed as universally applicable.

### EXAMPLE

### Example 1

The present example serves to validate herein disclosed technology using an experimentally acquired training dataset that demonstrates the ability of a ML model to identify biomarkers related to a specific task. The selected experimental task is the severity scoring of Obstructive Sleep Apnea-Hypopnea (OSA) from PSG signals. The exemplary framework provides a comparison between samples to maximize the interpretability of the results.

This exemplary framework of the technology is discussed hereinbelow with reference to the enclosed figures. The present example is meant to aid the reader in understanding the technological concepts more easily, but it is not meant to identify the most important or essential features thereof, nor is it meant to limit the scope of the present disclosure. The ML model of the present disclosure is not limited to the specific encoder architecture of the below example and relevant hyperparameters (including the latent space dimension, batch size, dropout, weight decay, and so on). It is understood that the ML model of the below example is a preferred embodiment of the ML model of the present disclosure.

### Machine Learning Model

FIG. 2 shows a model of the present example that is composed of three modules, namely, a Variational Autoencoder (VAE) module 10, a Generative Adversarial Network (GAN) module with a Multi-Layer Perceptron (MLP) as discriminator 50, and a classifier as scoring module 70, which are trained sequentially using a semi-supervised curriculum learning process with the objective of encoding the input data into a latent feature space maximizing the discriminability between samples of different OSA severity levels. The modules make of make use of a convolutional encoder 20 that encodes the input data into an embedding, the encoder latent space, referred to in the present example as (Ze).

The VAE 10 first estimates the mean (μ) and the standard deviation (σ) of the dataset distribution from Ze using dense layers to obtain a decoder latent space, referred to in the present example as (Zd). Then, Zd is decoded to derive a reconstructed version of the input data using a deconvolutional decoder. For the model of the present example, the decoder latent space (Zd) is obtained by the reparameterization trick classically used in VAEs, i.e., Zd=μ+σϵ=where ϵ is a random variable with Gaussian distribution. The loss function used to train the VAE 10 module is a combination of reconstruction loss and Kullback-Leibler divergence, as described in the following Equation:

$${ℒ}_{VAE} = 0.5 MSE ( output , input ) + 0.5 \frac{1}{b s} {\sum }_{i = 1}^{bs} − 0.5 {\sum }_{latent \_ dim} ( 1 + log ( σ ) − {μ}^{2} − σ ) ,$$

with bs the batch size and MSE the mean-squared error. As only Zd acquires the generative ability and follows a Gaussian distribution during the VAE training phase, another process may transfer these properties to Ze that is the purpose of the GAN module.

The GAN exploits the encoder as a generator and discriminates Zd (real distribution) from Ze (fake distribution) using an MLP discriminator. By considering Zd as the real latent representation and Ze as the fake one, the training of the GAN module will force the encoder to directly generate a latent vector Ze that mimics the properties of Zd.

The GAN module of the present example includes a generator that generates fake data 51 as close as possible to real data 52 and a discriminator 50 that differentiates between the fake data 51 and the real data 52. The generator is the encoder shared with the VAE 10 and the discriminator consists of a 3-layer MLP each of them using leaky ReLU activation function with a negative slope of 0.2 and batch normalization. The output activation function is a sigmoid function. The loss function of the generator is a weighted sum of the VAE loss and the mean of correct predictions by the discriminator within a batch:

$${ℒ}_{g e n e r a t o r} = 0.2 \cdot {ℒ}_{VAE} + 0.8 \cdot \frac{1}{b s} {\sum }_{i = 1}^{bs} ( 1 - {fake}_{i} ) )$$

with fake the output the discriminator when the fake latent representation (Ze) is given as input, which should be equal to 1 for an ideal generator. The loss function of the discriminator is the difference between the mean fake predictions and the mean real predictions:

$${L}_{discrimator} = \frac{1}{b s} {\sum }_{i = 1}^{b s} {fake}_{i} - \frac{1}{b s} {\sum }_{i = 1}^{b s} {real}_{i} ) ,$$

with real the output of the discriminator when the real latent representation (Zd) is given as input. For an ideal discriminator, real=1 and fake=0. The combination of both loss functions is described further below.

The MLP uses the features extracted by the encoder in Ze to classify the hypoxic burden, the arousal events and the respiratory event duration with a unique single-layer perceptron. In this way, the classifier module forces the encoder to output a latent vector where trials presenting a low level of severity are maximally distant from trials exhibiting a high level of severity. In the present example, the level of severity is characterized by the hypoxic burden (i.e., the area over the curve of the SAO2 signal), the arousal events (i.e., the presence or not of an arousal occurring just after the OSA) and the duration of the respiratory event. For clarity and simplicity of the present example, two severity levels are defined by computing the median values of each severity features across trials and considering trials below the median value as low severity level trials and trials above the median values as high severity level trials, except for the arousal events which are binary. Finally, four levels of severity are obtained including: (1) Very Low (low severity level on all three features), (2) Low (high level on 1 feature), (3) High (high level on 2 features), (4) Very High (high level on all three features).

For the encoder 20 to perform a major part of the classification task instead of the classifier layer itself, the classifier block is designed as a single-layer perceptron that only performs a linear combination of the 128 latent vector values to provide a probability for the sample to be of each class using a softmax activation function. The training process as described below is done separately for each severity feature with the loss function being a binary cross-entropy loss: [symbol] =BCE (predicted, target).

### Biosignal Dataset

The dataset built for the present example consists of PSG data of 72 subjects who had undergone in-lab PSG (≥8 hours). The recordings, realized in the Sleep Laboratory, Centre Hospitalier Universitaire Saint-Pierre (CHU St-Pierre), Brussels, Belgium, have been manually annotated by clinicians to identify sleep stages, apnea and hypopnea events and arousal events according to international guidelines.

All the selected subjects exhibited excessive obstructive respiratory events (apnea or hypopnea) during the night, with at least AHI>5. Sleep onset was determined when the first epoch of sleep occurs. A preliminary sleep questionnaire was performed and the protocol CE/22-03-03 was approved by the local ethical committee of the CHU St-Pierre.

The PSG sensors are composed of 6 EEG electrodes, 2 Electro-oculograph (EOG) electrodes (EOG1 under the left eye and EOG2 above the right eye), thoracic and abdominal belts (VTH and VAB) to monitor respiratory motions, an Electrocardiogram (ECG) sensor, a pulse oximetry sensor recording the pulse rate (PR) and the oxygen saturation (SAO2), and a pressure probe measuring the nasal airflow (NAF2P) The raw signals were recorded at 200 Hz and down sampled at Hz for storage requirements using Medatec Brainnet Winrel 5.0 system. The data were then converted to Python friendly files using the MNE-Python package, which was used to pre-process the signals.

As the analysis focuses on the differences between apnoeic events, the studied database consists of OSA trials only, each of them corresponding to a 70 sec segment extracted from the manually labelled signals and starting 4 seconds before an OSA event. Each trial is composed of 23 channels and 3001 timestamps. The selected EEG signals were the 3 left-hand side electrodes, a frontal (FP1), a central (C3) and an occipital (O1), with the reference electrode being placed just above the nasion and the derivations being performed with a right mastoid electrode. The 3 right-hand side electrodes were not analysed for clarity and simplicity of this proof-of-concept research. The signals have been pre-processed for artifacts removal, subject exclusion and addition of Pulse Rate Variability (PRV) signal from the PR signal and phase shift (Pshift) signal from the VAB and VTH signals. The normalization has been performed by channel independently as a z-score normalization with clamping in the [−3; 3] range.

After the pre-processing phase, the final dataset is composed of 6992 OSA trials from 70 subjects divided into a training set of 4670 trials from 48 subjects, namely the trainset, and a validation set of 23 trials from the 12 remaining subjects, namely the test set.

### Training

The training process of the proposed model consists in a semi-supervised curriculum learning framework.

Every block of the above-described architecture is trained separately, and the initialisation of the following block training process is done using the updated weights obtained at the end of the previous stage. The VAE 10 and the GAN modules are trained with non-supervised learning, while the MLP classifier is trained in a supervised manner, making the whole model training semi-supervised. The VAE 10 module has been trained using a random initialization until convergence. Then, the GAN module has been trained by initializing the generator with the best weights of the encoder obtained during the VAE training phase and the discriminator has been randomly initialized.

At each batch, the discriminator is first trained by freezing the generator and using the loss function of the discriminator described above, then the generator is trained by freezing the discriminator and using the corresponding loss function. Every 15 epochs, the updated network is used in inference to compute a new Zd vector given as real input for the 15 following epochs in order to avoid the deterioration of the “real” space to be responsible for the increase of the GAN performance.

Finally, the MLP classifier 70 module is initialised with the weights of the best generator previously obtained and the single-layer perceptron is randomly initialized. In the philosophy of curriculum learning, the classifier is trained on each severity feature sequentially, starting with the low vs. high severity classification on the hypoxic burden, then on the arousal events and finally on the event duration. On the hypoxic burden, the learning rate was set to 10⁻³ and, once every 5 epochs, the whole model was trained using a global loss combining the three modules:

$${ℒ}_{global} = \frac{1}{3} {ℒ}_{V A E} + \frac{1}{3} \frac{1}{b s} {\sum }_{i = 1}^{b s} ( 1 - {fake}_{i} ) + \frac{1}{3} {ℒ}_{c lassifier} .$$

On the arousal events, the learning rate was set to 5·10⁻⁴, the global training was performed every 5 epochs and, once every 2 epochs, the classification has been performed on both the hypoxic burden and the arousal events using a weighted sum of both losses: [symbol] =0.5· [symbol] +0.5· [symbol] .

On the respiratory event duration, the learning rate was set to 2·10⁻⁴, the global training was performed every 5 epochs and, twice every 3 epochs, the classification has been performed on all the severity features using a weighted sum of all classification losses:

$${L}_{{classifiers}_{3}} = \frac{1}{3} \cdot {ℒ}_{hypoxic burden} + \frac{1}{3} \cdot {ℒ}_{arousal event} + \frac{1}{3} \cdot {ℒ}_{duration} .$$

### Explainability

To explain how the model makes its decisions, an interpretability-focused approach is proposed consisting in highlighting the similarities and differences between samples from different parts of the encoded latent space. In fact, the latent vector encoded by the encoder (Ze) can be considered as a projection of the input data into a 128-dimensional space. This space being non-sparse owing to the Gaussian and generative properties, can be navigated through in any direction to explore the specificity of each region. As the feature of interest of the present example is the severity of the OSAs a subject undergoes, the principal direction for where the severity is encoded is searched by performing a Linear Discriminant Analysis (LDA) on Ze that maximizes the discrimination between the four classes of severity. The result of this process is a vector giving the direction of the severity encoding, namely the severity direction. By comparing input samples along the severity direction, the channels that vary the most as well as the time windows that are the most affected by the OSAs severity can be highlighted. By analysing non-EEG channels, the model that actually looks at the important features for severity scoring can be validated. By analysing EEG channels, the best biomarkers of OSA in the EEG signals can be identified.

### Results

The results of this model, essentially qualitative, can be divided in two parts: 1) the severity scoring efficiency and 2) the EEG biomarkers identification.

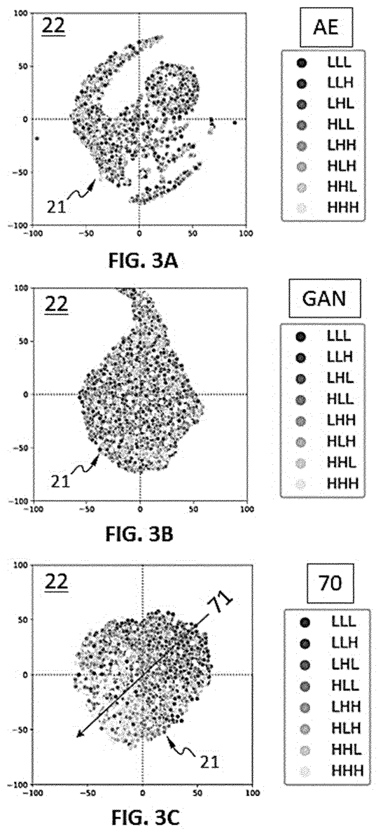

In FIG. 3 (FIG. 3A-FIG. 3C), the evolution of the latent space distribution can be observed across the different training phases of the VAE 10 module, the GAN module and the MLP 70 module, allowing the qualitative evaluation of how Ze acquires the required properties. For illustration purpose, the 128-dimensional latent space has been projected to a 2D space using the t-distributed stochastic neighbour embedding (t-SNE) transform. In the legend, each letter of “h”, “a” and “d” represents a severity feature: “hypoxic burden”, “arousal event”, and “duration of the respiratory event”, respectively. The “L” means “Low-level severity”, the “H” means “High-level severity”.

As shown in FIG. 3A, the training process of the VAE 10 module leads to a sparse encoder latent space (Ze). As further shown in FIG. 3B, the training process of the GAN module leads to a non-sparse Ze getting closer to a Gaussian distribution, but with the samples of different severity scores randomly distributed. The training process of the classifier module leads to a non-sparse, quasi-Gaussian Ze where the samples of similar severity level tend to be gathered together and separated from samples of different severity levels, as illustrated in FIG. 3C.

From this well-designed latent space, a LDA aiming at classifying the four severity levels has been performed. With the classifier module trained, this LDA reaches a mean accuracy of 54.0% (train set) and 48.8% (test set), and a mean F1-score of 56.5% (train set) and 48.4% (test set). The direction of highest severity score variance, namely the severity direction, is represented with an arrow 71 in FIG. 3C and is responsible for 78.14% of the explained variance. Specifically, the severity scale 71 represents the severity direction obtained by sorting the trials based on their severity score. This ability to estimate the severity score from a trial representation in Ze is the first proof of the relevance of the proposed framework in severity scoring task. By navigating along the severity direction, the OSA trials can be sorted by severity score to generate a severity scale and compare the trials depending on their position on this scale. The position of a latent vector (coming from a specific trial) within this scale determines the final severity score that may be provided to a medical professional as output—as discussed further below.

In FIG. 4 (FIG. 4A-FIG. 4D), the biomarkers identification is shown by comparing the power signal, by channel, of the OSA trials sorted by severity score (E [0,1]) along the severity direction obtained using LDA. FIG. 4A provides a summary of the influence of the severity score on each PSG channels based on their power signal. This comparison is performed by computing the mean power difference of each channel separately as described in the following Equation:

$${P}_{{difference}_{c [ i ] dist [ j ]}} = \frac{1}{N - j} {\sum }_{k = 0}^{N - j} {P}_{c [ i ] t [ k + j ]} - {P}_{c [ i ] t [ k ]} ,$$

$${P}_{{difference}_{c [ i ]}} = \frac{1}{N} {\sum }_{j = 0}^{N} {P}_{{difference}_{c [ i ] dist [ j ]}} ,$$

with trials being sorted based on their severity score, N the number of trials, t the trial number, c the channel and dist being the distance on the severity scale. The second operation allowing the evaluation of the severity scoring efficiency consists in identifying PSG channels and time windows that vary the most with the severity score. The non-EEG PSG are used to evaluate the consistency between clinical studies and the proposed framework, while the EEG channels allow the biomarkers discovery. In FIG. 4A and FIG. 4B, the high positive power difference on the SAO2 signal suggests deeper and/or longer desaturations of severe OSA trials. The high negative power difference on the EOG signal is consistent with available literature.

Furthermore, FIG. 4C shows that the SAO2 effect mainly appears during the respiratory events (beginning of the trial) with a spurious peak effect around seconds after the start of the event (note that OSA event starts after 4 sec as described above). The aforementioned results provide the desired second proof that the proposed framework actually extracts severity information. The EEG biomarkers identification task is based on the information provided by FIG. 4D and FIG. 4E where it can be observed that the central electrode (C3) is the most affected by the severity of the respiratory event in the 2-8 Hz frequency range, this effect being maximal in the 5-25 sec trial time window (corresponding to the mean respiratory event duration). The occipital electrode (O1) also varies with the severity score in the 2-8 Hz frequency range, but the frontal one (FP1) seems not to be influenced by the OSA severity. All the identified effects correspond to a decrease in the EEG power in parietooccipital regions when the severity score increases which can be interpreted as a reduced brain activity during severe OSA events.

### Example 2

The following example illustrates an implementation of the technology disclosed herein through the inference of a trained ML model on subject-specific data for individuals experiencing varying degrees of sleep disorders that could potentially impact their health and reported well-being. In this exemplary implementation, the method proceeds by utilizing a severity score generated by an ML model described in Example 1, trained on data related to Obstructive Sleep Apnea-Hypopnea (OSA).

As a baseline reference, the assessment of subjects is conducted in accordance with the standard medical scoring method using the Apnea-Hypopnea Index (AHI), determined by the number of disturbances occurring overnight as assessed by a medical professional. The outcomes of the AHI scoring are compared with the severity scoring generated by the trained ML model. Subsequently, the subject's medical condition is evaluated by a medical professional based on a combination of the AHI and severity score to assist in identifying the necessity and type of medical treatment for the subjects.

In the following cases, the predetermined threshold for AHI is set at 15, wherein AHI<15 is considered low to normal, and AHI>15 is considered elevated. Furthermore, the predetermined threshold for the generated severity scores is set at 0.25, wherein a severity score <0.25 is considered low to normal, and a severity score >0.25 is considered elevated.

### First Case (Elevated AHI/Elevated Severity Score)

In the first case, a 50-year-old male patient underwent assessment revealing a markedly elevated AHI of 35, firmly placing him within the severe category for OSA according to conventional diagnostic criteria.

The subject has a Body Mass Index (BMI) of 32, indicative of obesity, and reported severe daytime sleepiness, a hallmark symptom of OSA. Additionally, he presented with significant cardiovascular comorbidities, notably hypertension, evidenced by average blood pressure readings of 145/95 mmHg, heightening his susceptibility to OSA-related complications.

The severity score obtained through the herein described ML based scoring method mirrors the severity of the subject's condition. This alignment underscores a critical clinical state characterized not only by the frequency of respiratory events but also by aberrant breathing patterns. Polysomnography (PSG) data revealed pronounced heart rate variations during OSA events, alongside significant asynchrony between thoracic and abdominal movements post-events, indicative of compromised respiratory efforts during sleep.

Concurrently high AHI and severity scores validate the importance for immediate and intense intervention. This comprehensive assessment mandates consideration of continuous positive airway pressure (CPAP) therapy as a primary treatment modality. Additionally, the subject receives guidance on weight management strategies and sleep posture adjustments to mitigate the occurrence of respiratory events.

While no specific alert is generated for the clinician, the scoring system highlights the pulse rate variation (PRV) signal and phase shift between thoracic and abdominal movements as noteworthy biomarkers. These biomarkers serve as additional avenues for deeper exploration of the subject's needs, potentially prompting referral to a cardiologist for monitoring of further cardiac risks. This further underscores the system's capacity to capture the multifaceted nature of OSA severity, potentially guiding clinicians towards interventions that address both the frequency of respiratory disruptions and their physiological repercussions, ensuring more holistic subject care.

### Second Case (Normal AHI/Elevated Severity Score)

In the second case, a 42-year-old male patient exhibited a notable hypoxic burden during sleep, characterized by a significant proportion of respiratory events marked by desaturations of at least 4%. Particularly severe episodes occurred predominantly between the 4th and 6th hour of sleep, coinciding with an elevated AHI of approximately 30, while the AHI during the remaining sleep period averaged around 5, resulting in a final AHI of 10.

Despite having a normal BMI of 24 and no daytime excessive sleepiness, this subject presented with risk factors including a smoking habit and moderate hypertension, evidenced by an average blood pressure reading of 150/90 mmHg, alongside complaints of snoring.

Conventional clinical evaluation, considering the low AHI, might overlook this subject, potentially resulting in minimal recommendations from the clinician, primarily focused on reducing smoking. However, the proposed scoring method effectively alerts the clinician to the subject's heightened severity score, prompting a need to treatment.

Moreover, the proposed solution emphasizes the oxygen saturation (SAO2) signal as a pivotal biomarker contributing to the severity of respiratory events, particularly during critical periods between the 4th and 6th hour of sleep.

With this additional insight, the clinician reevaluates the subject's case, delving deeper into PSG data captured during these critical events, alongside consideration of comorbidities.

Subsequently, based on the clinician's re-evaluation, the recommended course of action involves a multifaceted approach, including smoking cessation and the implementation of positional therapy utilizing a positional belt. This comprehensive treatment strategy aims to address not only the immediate concerns but also mitigate long-term risks associated with sleep-disordered breathing.

### Third Case (Elevated AHI/Normal Severity Score)

In the third case, a 55-year-old female subject presented with an elevated AHI of 28, primarily attributed to a series of brief respiratory events lasting 5 to 10 seconds. These findings aligned with the criteria for moderate to severe OSA under traditional diagnostic standards. The subject has a BMI of 30 and reported mild daytime sleepiness, yet lacked significant cardiovascular comorbidities, maintaining an average blood pressure of 130/85 mmHg.

Despite the elevated AHI, the subject's overall hypoxic burden during sleep remained minimal, with infrequent episodes of SAO2 signal dipping below 90%. Notably, respiratory events occurred uniformly throughout the night, and thoracic and abdominal movements remained in phase post-event. Furthermore, her pulse rate demonstrated little variation during OSA events.

While conventional evaluation might prompt consideration for continuous positive airway pressure (CPAP) therapy based on the moderate-severe AHI level, the ML generated severity scoring generated a low severity score for this subject. This alerted to the mild nature of her hypoxic episodes, nuanced breathing patterns, and minimal heart rate variability during events.

Though none of the biomarkers of interest were flagged by the system, the emphasis on the brevity of respiratory events provided valuable insight. This nuanced analysis suggested that despite an AHI surpassing intervention thresholds, the actual severity of her condition may not mandate immediate aggressive treatment.

Armed with this insight, the clinician opted against immediate CPAP therapy, instead focusing on tailored lifestyle modifications. Recommendations included weight loss initiatives, increased physical activity, and enhancements to sleep hygiene practices. The subject was scheduled for regular follow-up evaluations to monitor any changes in her condition warranting a reassessment of treatment needs.

This scenario underscored the significance of the proposed scoring method in offering a more nuanced and subject-centric approach to OSA management. By transcending the AHI as the sole determinant of therapy, clinicians could devise personalized treatment strategies that better aligned with individual physiological nuances and risk profiles, ultimately optimizing subject care outcomes.

### CONCLUSION

The above cases illustrate the advantages of utilizing severity scoring as a supportive diagnostic tool for assessing the severity of sleep-related disorders. Specifically, the herein described severity scoring can be used to identify the need for medical treatment in patients who might have been overlooked using the standard medical scoring method (AHI). Alternatively, the herein described severity scoring can help mitigate the impact of medical treatment prescribed to patients who score higher on the standard medical method (AHI). Additionally, the identification of specific severity features or biomarkers identified by the severity scoring method can enable a medical professional to better assess the correct treatment method for addressing the specific sleep disorder, resulting in a more personalized treatment.

1. A computer-implemented method for assessing the severity of a sleep disorder of a subject, comprising the steps of:
receiving biosignal data associated with a plurality of trials representing different levels of severity of the sleep disorder, wherein the biosignal data includes a plurality of severity features variably impacting the severity of the sleep disorder;
training a machine learning model to encode the biosignal data from the trials into a plurality of latent vectors characterizing the severity of the sleep disorder, utilizing the severity features, and arranging the latent vectors in a latent space to increase discriminability between latent vectors representing different levels of severity;
operating the trained machine learning model on subject biosignal data, obtained from the subject, to generate a severity score indicative of the severity of the sleep disorder of the subject, determined from a position of the corresponding latent vectors relative to the positions of each latent vector within the latent space; and
generating a report comprising at least the severity score for assessing the severity of the sleep disorder of the subject;
wherein the machine learning model comprises an auto-encoder module including an encoder and a decoder, a generative adversarial network module including a discriminator, and a scoring module;
wherein the auto-encoder module is configured to encode distinct features contained in the biosignal data into the plurality of latent vectors such that the biosignal data can be reconstructed;
wherein the generative adversarial network module is configured to transfer non-sparsity and generative properties from a reference distribution to the latent space through an adversarial learning process, whereby the discriminator is trained to distinguish between real data, sampled from the reference distribution, and fake data, sampled from the latent space generated by the encoder aimed at deceiving the discriminator; and
wherein the scoring module is configured to estimate values of the severity features based on the position of each latent vector within the latent space.

2. The method according to claim 1, wherein the severity score is generated by sorting the plurality of latent vectors along a severity direction, thereby generating a severity scale that represent a multi-dimensional direction of the latent space along the plurality of latent vectors, and assigning a severity score for the trial based on the position of the corresponding latent vector along the severity scale.

3. The method according to claim 1, wherein the machine learning model is configured to arrange the latent vectors in the latent space to maximise a discriminability between the latent vectors representing different levels of severity by assigning maximally opposite values to the latent vectors that are furthest apart in the latent space.

4. The method according to claim 1, further comprising generating an alert if the severity score exceeds a predetermined threshold.

5. The method according to claim 1, wherein the report further comprises information about the severity features that are impacting the severity score of the sleep disorder of the subject.

6. The method according to claim 1, further comprising identifying one or more biomarkers that are indicative of the severity of the sleep disorder of the subject from the severity features; wherein the report further comprises the identified biomarkers.

7. The method according to claim 1, wherein the machine learning model is operable to identify one or more severity features included in the biosignal data that have an impact on the severity of the sleep disorder based on the plurality of latent vectors.

8. The method according to claim 1, wherein the latent space or reference distribution has a Gaussian distribution.

9. The method according to claim 1, wherein the latent space or reference distribution has a reconstructive property.

10. The method according to claim 1, wherein the auto-encoder module comprises a variational auto-encoder or an adversarial auto-encoder.

11. The method according to claim 1, wherein the auto-encoder module is trained with a loss function that includes at least one of a reconstruction loss, an adversarial loss, or a Kullback-Leibler divergence.

12. The method according to claim 1, wherein the generator of the generative adversarial network module is trained with a loss function including a weighted sum of the auto-encoder loss and the mean of correct predictions by the discriminator or a binary cross-entropy loss within the biosignal data, or the discriminator of the generative adversarial network module is trained on the difference between the mean fake predictions and the mean real predictions or binary cross-entropy loss.

13. The method according to claim 1, wherein the discriminator of the generative adversarial network module comprises a multilayer perceptron, wherein each layer of the multilayer perceptron is configured to apply a rectifier activation function with a negative slope and batch normalization.

14. The method according to claim 1, wherein the scoring module comprises at least one of a classifier or a regressor.

15. The method according to claim 1, wherein the scoring module is based on a threshold transfer function.

16. The method according to claim 1, wherein the biosignal data includes at least one of an electroencephalogram, an electrocardiogram, an electrooculogram, an electromyography, a photoplethysmogram, skin conductance, breath, heartrate, respiration airflow, or oxygen saturation data.

17. A system for assessing a severity of a sleep disorder of a subject, the system comprising a memory and a processor in communication with the memory, wherein the processor is configured to:
receive biosignal data associated with a plurality of trials representing different levels of severity of the sleep disorder, wherein the biosignal data includes a plurality of severity features variably impacting the severity of the sleep disorder;
train a machine learning model to encode the biosignal data from the trials into a plurality of latent vectors characterizing the severity of the sleep disorder, utilizing the severity features, and arrange the latent vectors in a latent space to increase a discriminability between the latent vectors representing different levels of severity;
operate the trained machine learning model on subject biosignal data, obtained from the subject, to generate a severity score indicative of the severity of the sleep disorder of the subject, determined from a position of the corresponding latent vectors relative to the positions of each latent vector within the latent space; and
generate a report comprising at least the severity score for assessing the severity of the sleep disorder of the subject;
wherein the machine learning model comprises an auto-encoder module including an encoder and a decoder, a generative adversarial network module including a discriminator, and a scoring module;
wherein the auto-encoder module is configured to encode distinct features contained in the biosignal data into the plurality of latent vectors such that the biosignal data can be reconstructed;
wherein the generative adversarial network module is configured to transfer the non-sparsity and generative properties from a reference distribution to the latent space through an adversarial learning process, whereby the discriminator is trained to distinguish between real data sampled from the reference distribution and fake data sampled from the latent space generated by the encoder aimed at deceiving the discriminator; and
wherein the scoring module is configured to estimate the values of the severity features based on the position of each latent vector within the latent space.

18. The system according to claim 17, wherein the processor is configured for performing a computer-implemented method for assessing the severity of a sleep disorder of a subject, comprising the steps of:
receiving biosignal data associated with a plurality of trials representing different levels of severity of the sleep disorder, wherein the biosignal data includes a plurality of severity features variably impacting the severity of the sleep disorder;
training a machine learning model to encode the biosignal data from the trials into a plurality of latent vectors characterizing the severity of the sleep disorder, utilizing the severity features, and arranging the latent vectors in a latent space to increase discriminability between latent vectors representing different levels of severity;
operating the trained machine learning model on subject biosignal data, obtained from the subject, to generate a severity score indicative of the severity of the sleep disorder of the subject, determined from a position of the corresponding latent vectors relative to the positions of each latent vector within the latent space; and
generating a report comprising at least the severity score for assessing the severity of the sleep disorder of the subject;
wherein the machine learning model comprises an auto-encoder module including an encoder and a decoder, a generative adversarial network module including a discriminator, and a scoring module;
wherein the auto-encoder module is configured to encode distinct features contained in the biosignal data into the plurality of latent vectors such that the biosignal data can be reconstructed:
wherein the generative adversarial network module is configured to transfer non-sparsity and generative properties from a reference distribution to the latent space through an adversarial learning process, whereby the discriminator is trained to distinguish between real data, sampled from the reference distribution, and fake data, sampled from the latent space generated by the encoder aimed at deceiving the discriminator; and
wherein the scoring module is configured to estimate values of the severity features based on the position of each latent vector within the latent space.

19. A computer program comprising instructions which, when the program is executed by a computer, perform the method according to claim 1.

20. A non-transitory computer-readable memory medium, which when loaded by at least one processor cause the at least one processor to perform the computer-implemented method according to claim 1.
